(19) United States
(12) Patent Application Publication
(10) Pub. No.: US 2023/0062911 A1
(43) Pub. Date: Mar. 2, 2023

(54) COMPOSITIONS AND METHODS FOR TREATING, PREVENTING, AND/OR AMELIORATING CHEMOTHERAPY-INDUCED PREMATURE OVARIAN FAILURE (POF)

(71) Applicant: Rutgers, The State University of New Jersey, New Brunswick, NJ (US)

(72) Inventor: Shuo Xiao, Piscataway, NJ (US)

(73) Assignee: Rutgers, The State University of New Jersey, New Brunswick, NJ (US)

(21) Appl. No.: 17/901,950

(22) Filed: Sep. 2, 2022

Related U.S. Application Data
(60) Provisional application No. 63/240,075, filed on Sep. 2, 2021.

(51) Int. Cl. A61K 38/22; A61P 15/08; A61K 31/015

(52) U.S. Cl. CPC A61K 38/22; A61K 31/015; A61P 15/08

(57) ABSTRACT

In one aspect, the present disclosure provides a method of treating, preventing, and/or ameliorating premature ovarian failure (POF) in a subject in need thereof, wherein the subject is a female cancer patient being administered at least one chemotherapeutic drug, the method comprising administering to the subject at least one c-Jun N-terminal Kinase (JNK) inhibitor. In certain embodiments, the JNK inhibitor is SP600125. In another aspect, the present disclosure provides a pharmaceutical composition comprising a JNK inhibitor.

### CROSS REFERENCE TO RELATED APPLICATION

The present application claims priority under 35 U.S.C. § 119(e) to U.S. Provisional Pat. Application No. 63/240,075, filed Sep. 2, 2021, which is hereby incorporated in its entirety herein.

### STATEMENT REGARDING FEDERALLY SPONSORED RESEARCH OR DEVELOPMENT

This invention was made with government support under grant number K01ES030014 awarded by the National Institutes of Health. The government has certain rights in the invention.

### BACKGROUND

Follicles represent the functional unit of the ovary and exist at many different developmental stages simultaneously within the ovary of a human female subject. Each follicle comprises a central oocyte surrounded by somatic follicular cells. Primordial follicles, which are follicles at the earliest stage of development, are finite from birth (i.e., non-renewable) and establish the ovarian reserve, which is indicative of the female reproductive life span.

Premature ovarian failure (POF) is a disease characterized by the depletion of primordial follicles before the age of 40, and is a major side effect of chemotherapy in childhood-age, adolescent, and young adult female cancer patients. The affected females are thus unable to bear children from a much younger age than most women, and may suffer additional symptoms, diseases, and/or disorders associated with insufficient ovarian hormones.

There is thus a need in the art for methods of treating, ameliorating, and/or preventing follicle loss and/or POF in a subject. The present disclosure addresses this need.

### BRIEF SUMMARY OF THE INVENTION

As described herein, the present invention relates to methods and compositions useful for treating, preventing, and/or ameliorating premature ovarian failure (POF) in subjects in need thereof. In certain embodiments, the methods and compositions comprise at least one c-Jun N-terminal Kinase (JNK) inhibitor (e.g. SP600125).

As such, in one aspect, the invention includes a method of treating, preventing, and/or ameliorating premature ovarian failure (POF) in a subject in need thereof, wherein the subject is a female cancer patient being administered at least one chemotherapeutic drug, the method comprising administering to the subject at least one c-Jun N-terminal Kinase (JNK) inhibitor.

In certain embodiments, the subject is pediatric, adolescent, or a young adult.

In certain embodiments, the cancer patient has at least one type of cancer selected from the group consisting of breast cancer, melanoma, prostate cancer, lung cancer, colorectal cancer, leukemia, brain tumor, spinal cord tumor, neuroblastoma, Whims tumor, lymphoma, rhabdomyosarcoma, retinoblastoma, and bone cancer.

In certain embodiments, the at least one chemotherapeutic drug is selected from the group consisting of doxorubicin (DOX), cisplatin, cyclophosphamide, carboplatin, oxaliplatin, methotrexate, and daunorubicin.

In certain preferred embodiments, the chemotherapeutic drug is DOX.

In certain embodiments, the POF comprises follicular atresia.

In certain embodiments, the POF comprises apoptosis in primordial follicle oocytes.

In certain embodiments, the POF comprises infertility or subfertility.

In certain embodiments, the POF comprises reduced synthesis and/or secretion of at least one steroid hormone.

In certain embodiments, the at least one steroid hormone is estrogen.

In certain embodiments, the at least one JNK inhibitor is selected from the group consisting of SP600125, AS601245, AS602801, JNK-IN-1, JNK-IN-8, ginsenoside Rg1, AV7, BI-78D3, pyridopyrimidinone 13, 4-quinolone analog 13c, and 4-phenylisoquinolone 11g.

In certain embodiments, the JNK inhibitor is SP600125.

In certain embodiments, the at least one chemotherapeutic drug and the JNK inhibitor are administered sequentially.

In certain embodiments, the at least one chemotherapeutic drug and the JNK inhibitor are administered simultaneously.

In certain embodiments, the at least one chemotherapeutic drug and the JNK inhibitor are co-formulated.

In another aspect, the invention provides a pharmaceutical composition comprising at least one JNK inhibitor, at least one chemotherapeutic drug, and a pharmaceutically acceptable carrier.

In certain embodiments, the at least one JNK inhibitor is selected from the group consisting of SP600125, AS601245, AS602801, JNK-IN-1, JNK-IN-8, ginsenoside Rg1, AV7, BI-78D3, pyridopyrimidinone 13, 4-quinolone analog 13c, and 4-phenylisoquinolone 11g.

In certain preferred embodiments, the JNK inhibitor is SP600125.

In certain embodiments, the at least one JNK inhibitor and the at least one chemotherapeutic drug are co-formulated.

In certain embodiments, the at least one chemotherapeutic drug is selected from the group consisting of DOX, cisplatin, cyclophosphamide, carboplatin, oxaliplatin, methotrexate, and daunorubicin.

### BRIEF DESCRIPTION OF THE FIGURES

The drawings illustrate generally, by way of example, but not by way of limitation, various embodiments of the present application.

FIG. 1 provides a graph illustrating the decrease in ovarian reserve over time in a human female subject.

FIG. 2A shows ovarian expression of γH2AX (DNA damage marker) and pTAp63α at 6 h post-PBS or DOX (10 mg/kg, i.p.). FIG. 2B provides a western blot showing expression of γH2AX and pTAp63α at 3 h and 6 h. The mobility shift of TAp63α indicated phosphorylation (i.e., p-TAp63α), which further indicated cell death initiation through apoptosis.

FIGS. 3A-3E show the in vivo treatment of DOX (10 mg/kg, i.p.) to 5-day old CD-1 female mice activated ovarian p53-mediated apoptosis JNK signaling pathways based on RNA sequencing analysis (FIG. 3A) and also the selective hyper-phosphorylation of JNK (FIG. 3B), the mechanism of JNK activation, in the oocytes of in primordial follicles at 6 h. FIG. 3C shows that JNK inhibitor SP600125 (50 mg/kg, i.p.) blocked the phosphorylation of TAp63α induced by DOX (10 mg/kg, i.p) and cisplatin (20 mg/kg, i.p.) in 5-day old mouse ovaries. FIG. 3D shows qRT-PCR results using whole neonatal ovaries from various treatment groups revealed that the co-treatment of SP600125 prevented the transcriptional induction of several established mediators during DOX-induced oocyte apoptosis, including Bax (BCL2 associated X), Puma (BCL2 binding component 3), and Noxa (Phorbol-12-myristate-13-acetate-induced protein 1, or Pmaipl). FIG. 3E shows that JNK inhibitor SP600125 prevented DOX-induced primordial follicle loss; ns: non-significant, **p<0.01, n=5 mice in each group.

FIGS. 4A-4B illustrate a similar DOX and SP600125 treatment experiment that was further performed using 21-day-old CD-1 female mice in order to mimic the effects of chemotherapy treatment in adolescent female cancer patients. FIG. 4A is a graph showing that DOX treatment remarkably reduced the numbers of all stages of ovarian follicles, including the stages of primordial, primary, secondary, and antral follicles. FIG. 4B illustrates ovarian cyclicity as examined by vaginal smear in the last 14 days of the 6-week post DOX / SP600125 treatment. The transition of various estrous cycle stages of three representative mice (a total of 6-8 mice in each group) are shown for each treatment group, including proestrus, estrus, metestrus, and diestrus.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

The term “excessive” as used herein refers to an event or process which occurs with a frequency greater than that which is reasonably considered necessary, normal, and/or desirable for the ordinary function of the event or process. In certain embodiments, the event or process is conception and/or pregnancy. In certain embodiments, the terms “excessive follicular atresia” and/or “excessive apoptosis in primordial follicle oocytes” may refer to a frequency of follicular atresia and/or apoptosis in primordial follicle oocytes which prevents and/or reduces the likelihood of conception and/or pregnancy.

The term “follicular atresia” as used herein refers to the hormonally controlled apoptotic process whereby a primordial or primary ovarian follicle is broken down and/or destroyed without oocyte release and/or ovulation.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The terms “infertility” and “subfertility” as used herein refer to a disease of the reproductive system that impairs one’s ability conceive a child without medical intervention. In certain embodiments, a subject may be considered infertile after failure to conceive a child after a period of 12 months of regular unprotected intercourse with a member of the opposite sex, wherein the failure to conceive is not attributable to improper functioning of the reproductive system of the member of the opposite sex in which the regular unprotected intercourse was engaged. In certain embodiments, an infertile subject requires medical intervention to achieve conception. Alternatively, a subfertile individual may achieve conception without medical intervention with an extended period of unwanted non-conception (i.e., longer than 12 months of regular unprotected intercourse).

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer’s solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington’s Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “premature ovarian failure” or “POF” as used herein refers the partial or total loss of reproductive and/or hormonal function of the ovaries in a subject below the age of 40, which may be attributable to follicular dysfunction (e.g., primordial follicle apoptosis) and/or early loss of eggs. In certain embodiments, a subject affected by POF may exhibit premature menopause (e.g., loss of menstruation and/or hot flashes). In certain embodiments, a subject affected by POF may exhibit sub-fertility or infertility.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

The term “primordial follicle” as used herein refers to a class of follicles formed in mammalian ovaries which comprise an oocyte surrounded by a single layer of flattened granulosa cells. A primordial follicle may remain quiescent, die by attrition, begin development with subsequent atresia, or begin development and release an oocyte followed by formation of a corpus luteum.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less. The term “substantially free of” can mean having a trivial amount of, such that a composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less, or about 0 wt%.

The terms “treat,” “treating,” and “treatment,” as used herein, means reducing the frequency or severity with which symptoms of a disease or condition are experienced by a subject by virtue of administering an agent or compound to the subject.

### Compositions

### Chemotherapeutics

In certain embodiments, the chemotherapeutic drug is

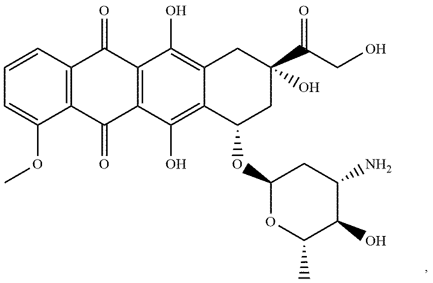

(8S,10S)-10-(((2R,4S,5R,6S)-4-amino-5-hydroxy-6-methyltetrahydro-2H-pyran-2-yl)oxy)-6,8,11-trihydroxy-8-(2-hydroxyacetyl)-1-methoxy-7,8,9,10-tetrahydrotetracene-5,12-dione (doxorubicin), or a salt or solvate thereof. In certain embodiments, the chemotherapeutic drug is

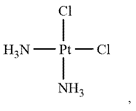

, cis-diammineplatinum (II) dichloride (cisplatin), or a salt or solvate thereof. In certain embodiments, the chemotherapeutic drug is

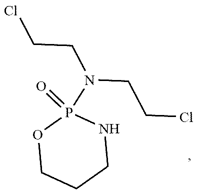

, 2-(bis(2-chloroethyl)amino)-1,3,2-oxazaphosphinane 2-oxide (cyclophosphamide), or a salt or solvate thereof. In certain embodiments, the chemotherapeutic drug is

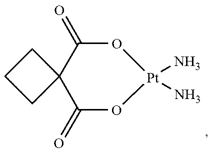

cis-(1,1-cyclobutanedicarboxylato)diammineplatinum(II) (carboplatin), or a salt or solvate thereof. In certain embodiments, the chemotherapeutic drug is

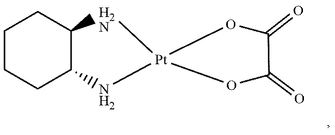

oxalato(trans-1-1,2-cyclohexanediamine)platinum(II) (oxaliplatin). In certain embodiments, the chemotherapeutic agent is

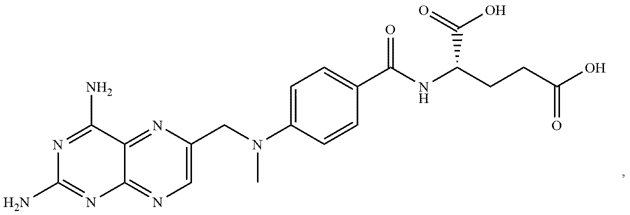

(4-(((2,4-diaminopteridin-6-yl)methyl)(methyl)amino)benzoyl)-L-glutamic acid (methotrexate). In certain embodiments, the chemotherapeutic drug is

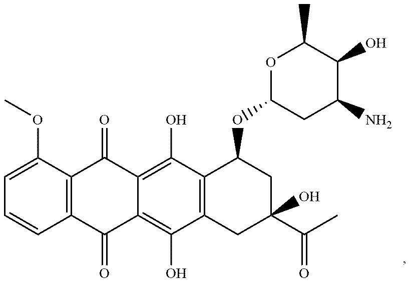

(8S,10S)-8-acetyl-10-(((2R,4S,5S,6S)-4-amino-5-hydroxy-6-methyltetrahydro-2H-pyran-2-yl)oxy)-6,8,11-trihydroxy-1-methoxy-7,8,9,10-tetrahydrotetracene-5,12-dione (daunorubicin).

### JNK Inhibitors

In certain embodiments, the JNK inhibitor is

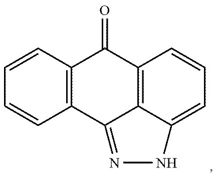

dibenzo[cd,g]indazol-6(2H)-one (SP600125), or a salt or solvate thereof. In certain embodiments, the JNK inhibitor is

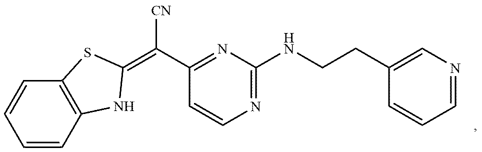

(Z)-2-(benzo[d]thiazol-2(3H)-ylidene)-2-(2-((2-(pyridin-3 -yl)ethyl)amino)pyrimidin-4-yl)acetonitrile (AS601245), or a salt or solvate thereof. In certain embodiments, the JNK inhibitor is

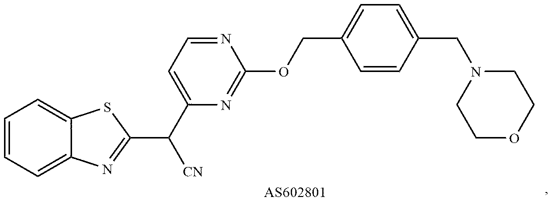

2-(benzo[d]thiazol-2-yl)-2-(2-((4-(morpholinomethyl)benzyl)oxy)pyrimidin-4-yl)acetonitrile (AS602801), or a salt or solvate thereof. In certain embodiments, the JNK inhibitor is

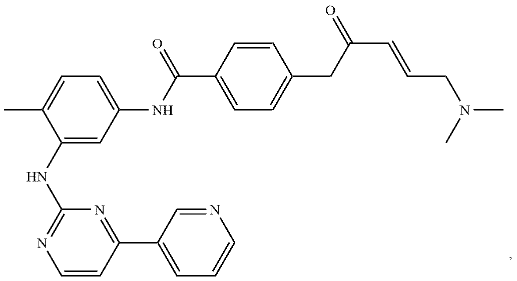

(E)-4-(5-(dimethylamino)-2-oxopent-3-en-1-yl)-N-(4-methyl-3-((4-(pyridin-3-yl)pyrimidin-2-yl)amino)phenyl)benzamide (JNK-IN-1), or a salt or solvate thereof. In certain embodiments, the JNK inhibitor is

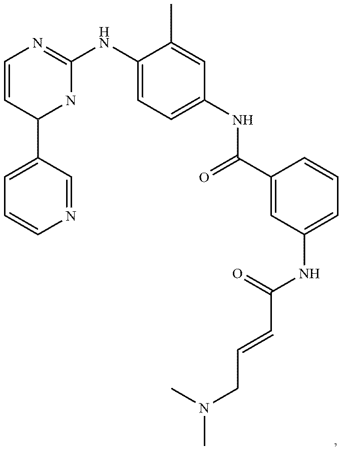

, (E)-3-(4-(dimethylamino)but-2-enamido)-N-(3-methyl-4-((4-(pyridin-3-yl)pyrimidin-2-yl)amino)phenyl)benzamide (JNK-IN-8), or a salt or solvate thereof. In certain embodiments, the JNK inhibitor is

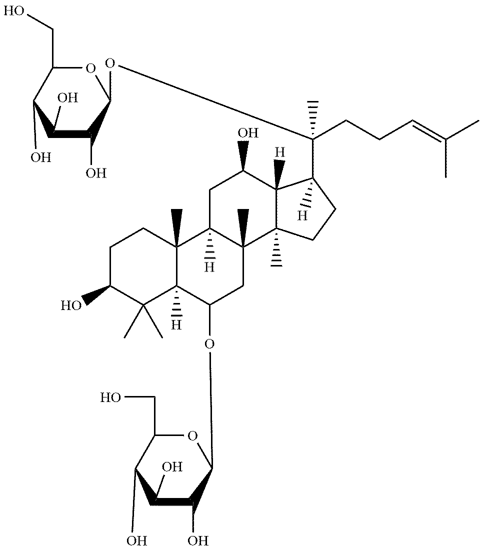

(2R,3R,4S,5S,6R)-2-(((3S,5R,8R,9R,10R,12R,13R,14R,17S)-3,12-dihydroxy-4,4,8,10,14-pentamethyl-17-((S)-6-methyl-2-(((2S,3R,4S,5S,6R)-3,4,5-trihydroxy-6-(hydroxymethyl)tetrahydro-2H-pyran-2-yl)oxy)hept-5-en-2-yl)hexadecahydro-1H-cyclopenta[a]phenanthren-6-yl)oxy)-6-(hydroxymethyl)tetrahydro-2H-pyran-3,4,5-triol (ginsenoside Rg1), or a salt or solvate thereof.

In certain embodiments, the JNK inhibitor is

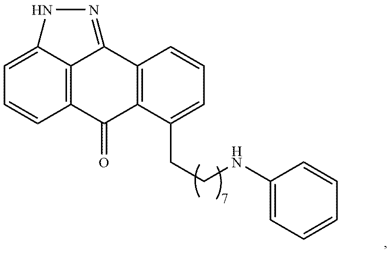

7-(8-(phenylamino)octyl)dibenzo[cd,g]indazol-6(2H)-one (AV7), or a salt or solvate thereof. In certain embodiments, the JNK inhibitor is

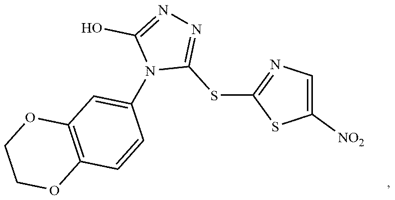

4-(2,3-dihydrobenzo[b][1,4]dioxin-6-yl)-5-((5-nitrothiazol-2-yl)thio)-4H-1,2,4-triazol-3-ol (BI-78D3), or a salt or solvate thereof. In certain embodiments, the JNK inhibitor is

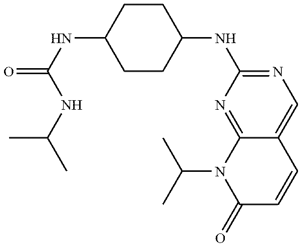 [SMILES: CC(C)NC(=O)NC1CCC(Nc2ncc3ccc(=O)n(C(C)C)c3n2)CC1]

, 1-isopropyl-3-(4-((8-isopropyl-7-oxo-7,8-dihydropyrido[2,3-d]pyrimidin-2-yl)amino)cyclohexyl)urea (pyridopyrimidinone derivative 13), or a salt or solvate thereof. In certain embodiments, the JNK inhibitor is

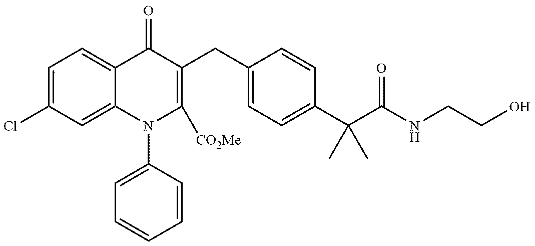 [SMILES: COC(=O)c1c(Cc2ccc(C(C)(C)C(=O)NCCO)cc2)c(=O)c2ccc(Cl)cc2n1-c1ccccc1]

methyl 7-chloro-3-(4-(1-((2-hydroxyethyl) amino)-2-methyl-1-oxopropan-2-yl)benzyl)-4-oxo-1-phenyl-1,4-dihydroquinoline-2-carboxylate (4-quinoline analog 13c), or a salt or solvate thereof. In certain embodiments, the JNK inhibitor is

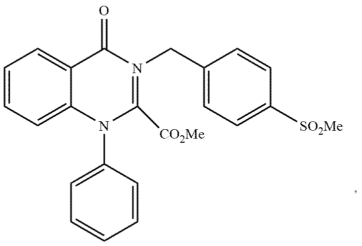

methyl 2-(4-(methylsulfonyl)benzyl)-1-oxo-4-phenyl-1,2-dihydroisoquinoline-3-carboxylate (4-phenylisoquinolone 11g), or a salt or solvate thereof.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H , ³H, ¹¹C _{>} ¹³C _{>} ¹⁴C _{>} ³⁶C1 _{>} ¹⁸F _{>} ¹²³I, ¹²⁵I, ¹³N _{>} ¹⁵N _{>} ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Methods and Kits

In one aspect, the present disclosure provides a method of treating, preventing, and/or ameliorating premature ovarian failure (POF) in a subject in need thereof, wherein the subject is a female cancer patient being administered at least one chemotherapeutic drug, the method comprising administering to the subject at least one c-Jun N-terminal Kinase (JNK) inhibitor.

In certain embodiments, the subject is of pediatric age (e.g., about 0 to about 18 years old). In certain embodiments, the subject is of adolescent age (e.g., about 10 to about 25 years old). In certain embodiments, the subject is a young adult (e.g., about 25 to about 40 years old). In certain embodiments, the subject is about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 years old.

In certain embodiments, the cancer patient has at least one type of cancer which is common in children. In certain embodiments, the cancer patient has at least one type of cancer selected from the group consisting of breast cancer, melanoma, prostate cancer, lung cancer, colorectal cancer, leukemia, brain tumor, spinal cord tumor, neuroblastoma, Whims tumor, lymphoma, rhabdomyosarcoma, retinoblastoma, and bone cancer.

In certain embodiments, the at least one chemotherapeutic drug is doxorubicin (DOX). In certain embodiments, the at least one chemotherapeutic drug is cisplatin. In certain embodiments, the at least one chemotherapeutic drug is cyclophosphamide. In certain embodiments, the at least one chemotherapeutic drug is carboplatin. In certain embodiments, the at least one chemotherapeutic drug is oxaliplatin. In certain embodiments, the at least one chemotherapeutic drug is methotrexate. In certain embodiments, the at least one chemotherapeutic drug is daunorubicin

In certain embodiments, the POF comprises follicular atresia. In certain embodiments the POF comprises apoptosis in primordial follicle oocytes. In certain embodiments, the POF comprises infertility or subfertility. In certain embodiments, the POF comprises reduced synthesis and/or secretion of at least one steroid hormone. In certain embodiments, the at least one steroid hormone is estrogen.

In certain embodiments, the at least one JNK inhibitor is SP600125. In certain embodiments, the at least one JNK inhibitor is AS601245. In certain embodiments, the at least one JNK inhibitor is AS602801. In certain embodiments, the at least one JNK inhibitor is JNK-IN-1. In certain embodiments, the at least one JNK inhibitor is JNK-IN-8. In certain embodiments, the at least one JNK inhibitor is ginsenoside Rgl. In certain embodiments, the at least one JNK inhibitor is AV7. In certain embodiments, the at least one JNK inhibitor is BI-78D3. In certain embodiments, the at least one JNK inhibitor is pyridopyrimidinone 13. In certain embodiments, the at least one JNK inhibitor is 4-quinolone analog 13c. In certain embodiments, the at least one JNK inhibitor is 4-phenylisoquinolone 11g.

In certain embodiments, the at least one chemotherapeutic drug and the JNK inhibitor are administered sequentially. In certain embodiments, the JNK inhibitor is administered before the chemotherapeutic drug. In certain embodiments, the JNK inhibitor is administered after the chemotherapeutic drug. In certain embodiments, the at least one chemotherapeutic drug and the JNK inhibitor are administered simultaneously. In certain embodiments, the at least one chemotherapeutic drug and the JNK inhibitor are co-formulated.

In one aspect, the present disclosure provides a kit comprising at least one JNK inhibitor, at least one chemotherapeutic drug, and optionally at least one applicator. In certain embodiments, the kit further comprises instructions for treating, preventing, and/or ameliorating POF in a subject in need thereof, wherein the subject is a female cancer patient to be administered the at least one chemotherapeutic drug. In certain embodiments, the instructions comprise instructions to administer to the subject the at least one c-Jun N-terminal Kinase (JNK) inhibitor.

### Pharmaceutical Compositions and Formulations

In another aspect, the present disclosure provides a pharmaceutical composition comprising at least one JNK inhibitor and a pharmaceutically acceptable carrier.

In certain embodiments, the at least one JNK inhibitor is SP600125. In certain embodiments, the at least one JNK inhibitor is AS601245. In certain embodiments, the at least one JNK inhibitor is AS602801. In certain embodiments, the at least one JNK inhibitor is JNK-IN-1. In certain embodiments, the at least one JNK inhibitor is JNK-IN-8. In certain embodiments, the at least one JNK inhibitor is ginsenoside Rg1. In certain embodiments, the at least one JNK inhibitor is AV7. In certain embodiments, the at least one JNK inhibitor is BI-78D3. In certain embodiments, the at least one JNK inhibitor is pyridopyrimidinone 13. In certain embodiments, the at least one JNK inhibitor is 4-quinolone analog 13c. In certain embodiments, the at least one JNK inhibitor is 4-phenylisoquinolone 11g.

In certain embodiments, the pharmaceutical composition further comprises at least one chemotherapeutic drug. In certain embodiments, the at least one JNK inhibitor and the at least one chemotherapeutic drug are co-formulated.

In certain embodiments, the at least one chemotherapeutic drug is doxorubicin (DOX). In certain embodiments, the at least one chemotherapeutic drug is cisplatin. In certain embodiments, the at least one chemotherapeutic drug is cyclophosphamide.

The invention provides pharmaceutical compositions comprising at least one compound of the invention or a salt or solvate thereof, which are useful to practice methods of the invention. Such a pharmaceutical composition may consist of at least one compound of the invention or a salt or solvate thereof, in a form suitable for administration to a subject, or the pharmaceutical composition may comprise at least one compound of the invention or a salt or solvate thereof, and one or more pharmaceutically acceptable carriers, one or more additional ingredients, or some combination of these. At least one compound of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., Recombumin®), solubilized gelatins (e.g., Gelofusine®), and other pharmaceutically acceptable salt solutions such as phosphates and salts of organic acids. Examples of these and other pharmaceutically acceptable carriers are described in Remington’s Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragranceconferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1 µg to about 7,500 mg, about 20 µg to about 7,000 mg, about 40 µg to about 6,500 mg, about 80 µg to about 6,000 mg, about 100 µg to about 5,500 mg, about 200 µg to about 5,000 mg, about 400 µg to about 4,000 mg, about 800 µg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 µg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound’s ability to perform its intended function, e.g., treating, preventing, or reducing a disease or disorder in a patient.

### Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pregelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer’s solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone. One acceptable vehicle for topical delivery of some of the compositions of the invention may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Pat. Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the compositions and/or formulations of the present invention may be, but are not limited to, short-term, rapid-offset, as well as controlled, for example, sustained release, delayed release and pulsatile release formulations.

The term “sustained release” is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term “delayed release” is used herein in its conventional sense to refer to a drug formulation that provides for an initial release of the drug after some delay following drug administration and that may, although not necessarily, include a delay of from about 10 minutes up to about 12 hours.

The term “pulsatile release” is used herein in its conventional sense to refer to a drug formulation that provides release of the drug in such a way as to produce pulsed plasma profiles of the drug after drug administration.

The term “immediate release” is used in its conventional sense to refer to a drug formulation that provides for release of the drug immediately after drug administration.

As used herein “short-term” refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein “rapid-offset” refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

Various embodiments of the present application can be better understood by reference to the following Examples which are offered by way of illustration. The scope of the present application is not limited to the Examples given herein.

### Example 1: Chemotherapeutic Agents Trigger Primordial Follicle Loss

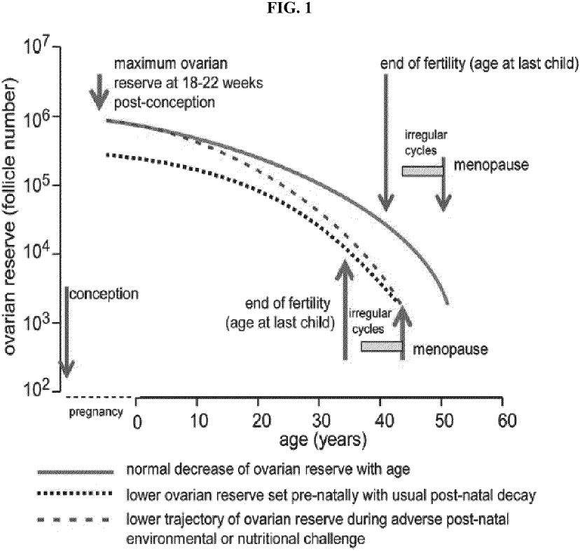

The ovarian reserve is defined at 18-22 weeks post-conception in a female. This reserve gradually decreases over time during the aging of a female, ultimately culminating in the end of fertility and menopause (FIG. 1). However, dietary and/or environmental factors including chemotherapy and/or radiation may contribute to development of premature ovarian failure (POF), which is characterized by an early onset of infertility and/or menopause.

In fact, doxorubicin (DOX) and cisplatin, common chemotherapeutic drugs, have been found to selectively activate c-Jun N-terminal kinase (JNK) in the oocytes of primordial follicles, thereby triggering primordial follicle death and leading to POF.

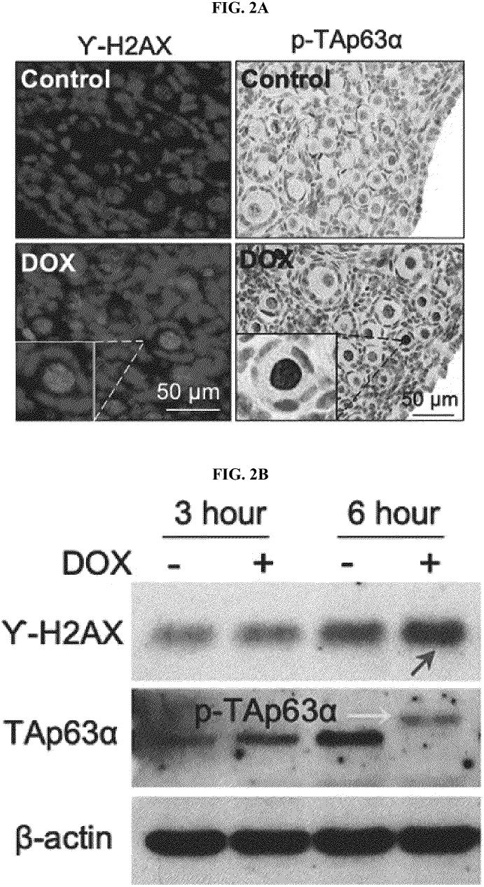

Expression of γ-H2AX, a well-characterized biomarker of DNA double strand breaks (DDSBs) as well as the major mechanism of DOX’s cytotoxicity on tumorous cells, was detected with administration of DOX. Results indicated that DOX significantly increased the expression of γ-H2AX in ovarian epithelial cells and oocytes (FIG. 2A) and the increased expression levels of γ-H2AX peaked at 6 h after DOX treatment (FIG. 2B, red arrow), indicating that DOX can induce DDSBs in oocytes of primordial follicles.

TAp63a functions to respond to DNA damage that results in its phosphorylation and subsequent oocyte apoptosis initiation in the primordial follicles. The expression of phospho-p63 was significantly induced in oocytes of primordial follicles 6 h post DOX treatment (FIG. 2A). In addition, western blot results showed that TAp63a was hyper-phosphorylated in DOX-treated ovaries at 6 h after DOX treatment (FIG. 2B).

### Example 2: Pharmacological Inhibition of JNK Prevents DOX-induced Primordial Follicle Loss

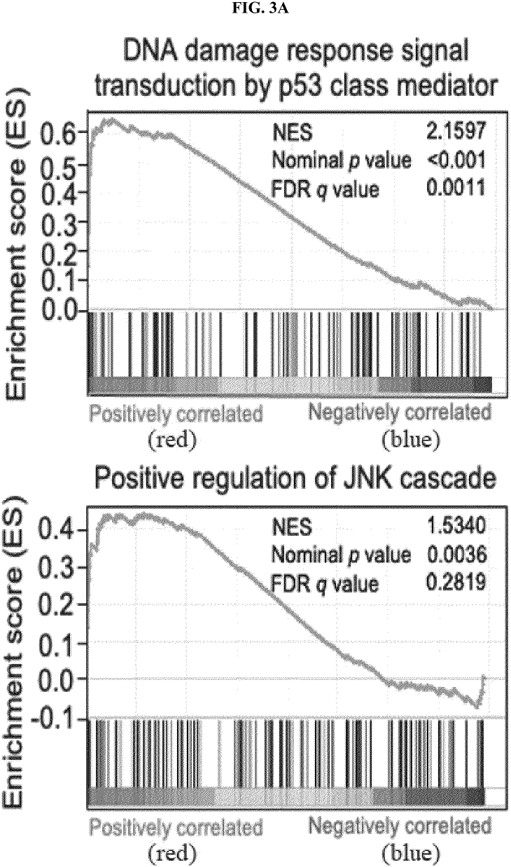

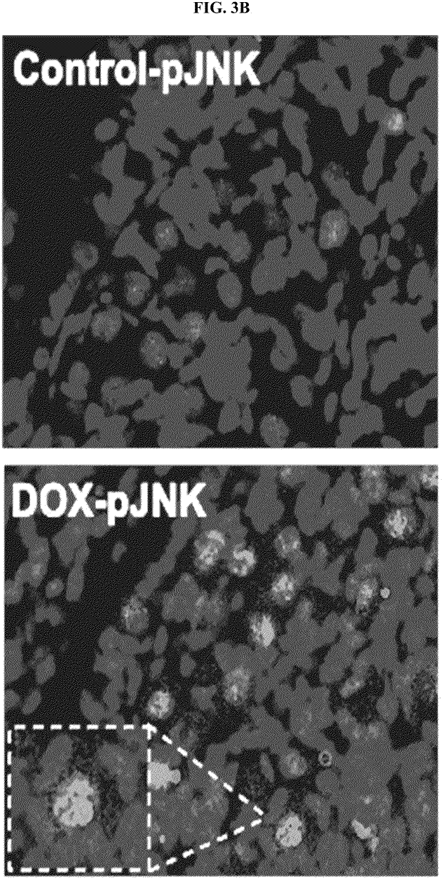

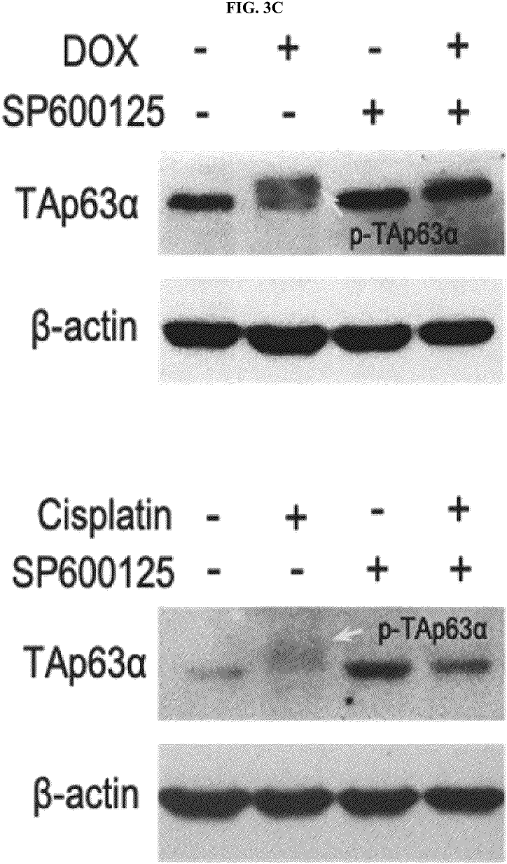

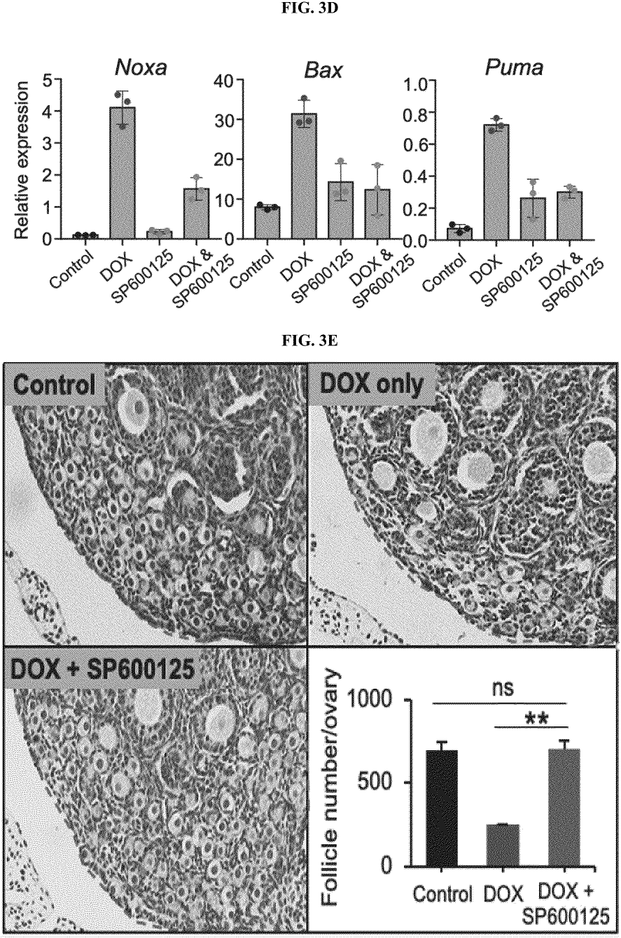

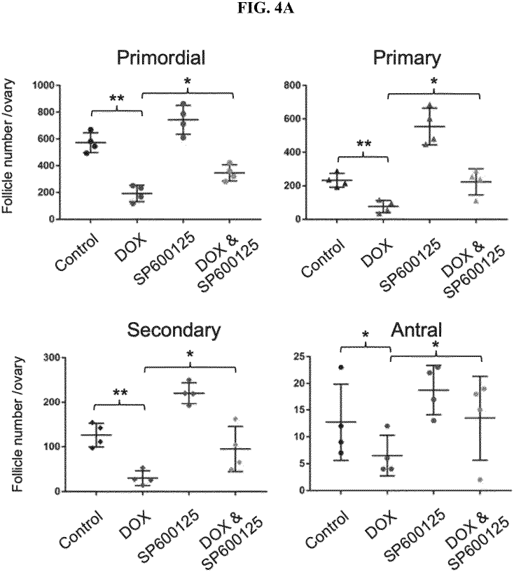

It has been demonstrated that chemotherapeutics (e.g., DOX and cisplatin) may specifically cause DNA damage and cell death in oocytes. Thus, the molecular mechanisms of DOX-induced primordial follicle oocyte apoptosis were investigated as described herein. Fiveday-old CD-1 female mice were intraperitoneally injected with vehicle or 10 mg/kg DOX and ovaries were harvested at 6 hours for RNA sequencing. The Gene Ontology analysis revealed that the JNK (c-Jun N-terminal kinase) signaling pathway was significantly changed after DOX treatment. Western blot and immunostaining results showed that there was a selectively increased phosphorylation of JNK, the mechanism of JNK activation, in the oocytes of primordial follicles (FIGS. 3A-3B).

Next, 5-day-old CD-1 female mice were intraperitoneally injected with 10 mg/kg DOX and 50 mg/kg SP600125, a specific JNK inhibitor. Western blot results showed that SP600125 effectively prevented DOX-induced hyper-phosphorylation of TAp63a, an oocyte-specific p53 family transcription factor that regulates oocyte apoptosis upon DNA damage (FIG. 3C). qRT-PCR results using whole neonatal ovaries from various treatment groups revealed that the co-treatment of SP600125 prevented the transcriptional induction of several established mediators during DOX-induced oocyte apoptosis, including Bax (BCL2 associated X), Puma (BCL2 binding component 3), and Noxa (Phorbol-12-myristate-13-acetate-induced protein 1, or Pmaipl) (FIG. 3D). In addition, the co-treatment of SP600125 significantly ameliorated DOX-induced primordial follicle loss (FIG. 3E).

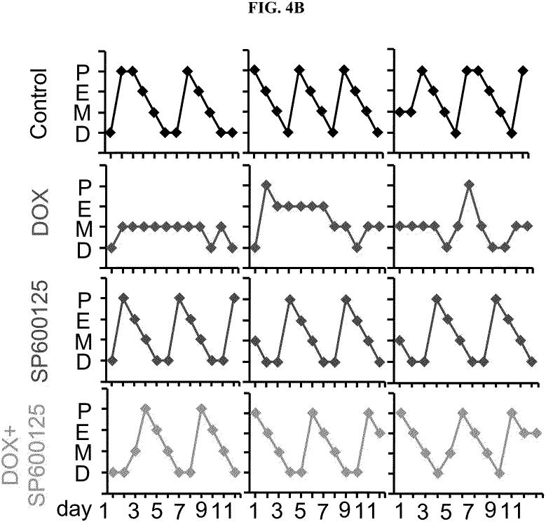

Another similar DOX and SP600125 treatment experiment was further performed using 21-day-old CD-1 female mice which mimics chemotherapy treatment in adolescent female cancer patients. Ovarian functions were examined at 6-weeks after treatment in order to evaluate the long-term effect of pharmacological JNK inhibition on female ovarian reserve and other ovary reproductive outcomes. The follicle counting results showed that DOX remarkably reduced the numbers of all stages of ovarian follicles, including the stages of primordial, primary, secondary, and antral follicles (FIG. 4A), confirming that DOX treatment induced POF. The treatment of JNK inhibitor SP600125 alone did not affect the numbers of all staged follicles (FIG. 4A), indicating that SP600125 treatment by itself had no ovarian toxicity. For the co-treatment group, SP600125 effectively prevented the reduction of all stages of follicles (FIG. 4A), demonstrating the protective effects of SP600125 on DOX-induced POF.

Ovarian cyclicity was next examined by the method of vaginal smear in the last 14 days of the 6-week post DOX / SP600125 treatment. The transition of various estrous cycle stages of three representative mice (a total of 6-8 mice in each group) are shown in FIG. 4B in each group, including proestrus, estrus, metestrus, and diestrus. Consistent to the follicle counting results in FIG. 4A, DOX interfered with mouse ovarian cyclicity and the majority of examined days were at metestrus stage, suggesting defective follicle development and hormone secretion (FIG. 4B). Mice treated with SP600125 had comparable ovarian cyclicity to the control group, indicating that SP600125 is not ovarian toxic. In the DOX / SP600125 co-treatment group, all mice had comparable ovarian cyclicity to the control group (FIG. 4B), demonstrating that the pharmacological inhibition of JNK can successfully prevent DOX-induced POF and associated ovarian functions long-term after chemotherapy; moreover, the ovarian protective effect of JNK inhibitor is applied to both neonatal and peripubertal mice that are corresponding to the pediatric and adolescent young female cancer patients.

Taken together, and without wishing to be bound by theory, these results demonstrate that JNK signaling critically contributes to chemotherapy-induced primordial follicle oocyte apoptosis and POF and that the pharmacological inhibition of JNK is a potential therapeutic regimen that can preserve young female cancer patients’ fertility and ovarian endocrine functions.

### Enumerated Embodiments

The following exemplary embodiments are provided, the numbering of which is not to be construed as designating levels of importance:

Embodiment 1 provides a method of treating, preventing, and/or ameliorating premature ovarian failure (POF) in a subject in need thereof, wherein the subject is a female cancer patient being administered at least one chemotherapeutic drug, the method comprising administering to the subject at least one c-Jun N-terminal Kinase (JNK) inhibitor.

Embodiment 2 provides the method of Embodiment 1, wherein the subject is pediatric, adolescent, or a young adult.

Embodiment 3 provides the method of any of Embodiments 1-2, wherein the cancer patient has at least one type of cancer selected from the group consisting of breast cancer, melanoma, prostate cancer, lung cancer, colorectal cancer, leukemia, brain tumor, spinal cord tumor, neuroblastoma, Whims tumor, lymphoma, rhabdomyosarcoma, retinoblastoma, and bone cancer.

Embodiment 4 provides the method of Embodiment 3, wherein the at least one chemotherapeutic drug is selected from the group consisting of doxorubicin (DOX), cisplatin, cyclophosphamide, carboplatin, oxaliplatin, methotrexate, and daunorubicin.

Embodiment 5 provides the method of Embodiment 4, wherein the chemotherapeutic drug is DOX.

Embodiment 6 provides the method of any of Embodiments 1-5, wherein the POF comprises follicular atresia.

Embodiment 7 provides the method of any of Embodiments 1-6, wherein the POF comprises apoptosis in primordial follicle oocytes.

Embodiment 8 provides the method of any of Embodiments 1-7, wherein the POF comprises infertility or subfertility.

Embodiment 9 provides the method of any of Embodiments 1-8, wherein the POF comprises reduced synthesis and/or secretion of at least one steroid hormone.

Embodiment 10 provides the method of Embodiment 9, wherein the at least one steroid hormone is estrogen.

Embodiment 11 provides the method of any of Embodiments 1-10, wherein the at least one JNK inhibitor is selected from the group consisting of SP600125, AS601245, AS602801, JNK-IN-1, JNK-IN-8, ginsenoside Rg1, AV7, BI-78D3, pyridopyrimidinone 13, 4-quinolone analog 13c, and 4-phenylisoquinolone 11g.

Embodiment 12 provides the method of Embodiment 11, wherein the JNK inhibitor is SP600125.

Embodiment 13 provides the method of any of Embodiments 1-12, wherein the at least one chemotherapeutic drug and the JNK inhibitor are administered sequentially.

Embodiment 14 provides the method of any of Embodiments 1-12, wherein the at least one chemotherapeutic drug and the JNK inhibitor are administered simultaneously.

Embodiment 15 provides the method of any of Embodiments 1-12 or 14, wherein the at least one chemotherapeutic drug and the JNK inhibitor are co-formulated.

Embodiment 16 provides a pharmaceutical composition comprising at least one JNK inhibitor, at least one chemotherapeutic drug, and a pharmaceutically acceptable carrier.

Embodiment 17 provides the pharmaceutical composition of Embodiment 16, wherein the at least one JNK inhibitor is selected from the group consisting of SP600125, AS601245, AS602801, JNK-IN-1, JNK-IN-8, ginsenoside Rg1, AV7, BI-78D3, pyridopyrimidinone 13, 4-quinolone analog 13c, and 4-phenylisoquinolone 11g.

Embodiment 18 provides the pharmaceutical composition of Embodiment 17, wherein the JNK inhibitor is SP600125.

Embodiment 19 provides the pharmaceutical composition of any of Embodiments 16-18, wherein the at least one JNK inhibitor and the at least one chemotherapeutic drug are co-formulated.

Embodiment 20 provides the pharmaceutical composition of any of Embodiments 16-19, wherein the at least one chemotherapeutic drug is selected from the group consisting of DOX, cisplatin, cyclophosphamide, carboplatin, oxaliplatin, methotrexate, and daunorubicin.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A method of treating, preventing, or ameliorating premature ovarian failure (POF) in a subject in need thereof, wherein the subject is a female cancer patient being administered at least one chemotherapeutic drug,
the method comprising administering to the subject at least one c-Jun N-terminal Kinase (JNK) inhibitor.

2. The method of claim 1, wherein the subject is pediatric, adolescent, or a young adult.

3. The method of claim 1, wherein the cancer patient has at least one type of cancer selected from the group consisting of breast cancer, melanoma, prostate cancer, lung cancer, colorectal cancer, leukemia, brain tumor, spinal cord tumor, neuroblastoma, Whims tumor, lymphoma, rhabdomyosarcoma, retinoblastoma, and bone cancer.

4. The method of claim 3, wherein the at least one chemotherapeutic drug is selected from the group consisting of doxorubicin (DOX), cisplatin, cyclophosphamide, carboplatin, oxaliplatin, methotrexate, and daunorubicin.

5. The method of claim 4, wherein the chemotherapeutic drug is DOX.

6. The method of claim 1, wherein the POF comprises follicular atresia.

7. The method of claim 1, wherein the POF comprises apoptosis in primordial follicle oocytes.

8. The method of claim 1, wherein the POF comprises infertility or subfertility.

9. The method of claim 1, wherein the POF comprises reduced synthesis and/or secretion of at least one steroid hormone.

10. The method of claim 9, wherein the at least one steroid hormone is estrogen.

11. The method of claim 1, wherein the at least one JNK inhibitor is selected from the group consisting of SP600125, AS601245, AS602801, JNK-IN-1, JNK-IN-8, ginsenoside Rg1, AV7, BI-78D3, pyridopyrimidinone 13, 4-quinolone analog 13c, and 4-phenylisoquinolone 11g.

12. The method of claim 11, wherein the JNK inhibitor is SP600125.

13. The method of claim 1, wherein the at least one chemotherapeutic drug and the JNK inhibitor are administered sequentially.

14. The method of claim 1, wherein the at least one chemotherapeutic drug and the JNK inhibitor are administered simultaneously.

15. The method of claim 1, wherein the at least one chemotherapeutic drug and the JNK inhibitor are co-formulated.

16. A pharmaceutical composition comprising at least one JNK inhibitor, at least one chemotherapeutic drug, and a pharmaceutically acceptable carrier.

17. The pharmaceutical composition of claim 16, wherein the at least one JNK inhibitor is selected from the group consisting of SP600125, AS601245, AS602801, JNK-IN-1, JNK-IN-8, ginsenoside Rg1, AV7, BI-78D3, pyridopyrimidinone 13, 4-quinolone analog 13c, and 4-phenylisoquinolone 11g.

18. The pharmaceutical composition of claim 17, wherein the JNK inhibitor is SP600125.

19. The pharmaceutical composition of claim 16, wherein the at least one JNK inhibitor and the at least one chemotherapeutic drug are co-formulated.

20. The pharmaceutical composition of claim 16, wherein the at least one chemotherapeutic drug is selected from the group consisting of DOX, cisplatin, cyclophosphamide, carboplatin, oxaliplatin, methotrexate, and daunorubicin.
